(19) United States
(12) Patent Application Publication
(10) Pub. No.: US 2014/0065685 A1
(43) Pub. Date: Mar. 6, 2014

(54) System and Method for Producing Ethanol and Biogas

(71) Applicants: Graig Rosenberger, Mullica Hill, NJ (US); Timur Dunaev, Philadelphia, PA (US)

(72) Inventors: Graig Rosenberger, Mullica Hill, NJ (US); Timur Dunaev, Philadelphia, PA (US)

(21) Appl. No.: 13/598,315

(22) Filed: Aug. 29, 2012

(51) Int. Cl. C12P 7/14; C12M 1/12

(52) U.S. Cl. 435/162; 435/297.1

(57) ABSTRACT

A system and process for producing ethanol and biogas. An incoming feedstock and water is directed to a preparation unit that frees sugar from the feedstock. The feedstock is directed to a fermenter that ferments the sugar to produce a beer that includes ethanol and feedstock. The beer is directed to a distillation unit which separates the ethanol from the feedstock and produces ethanol and whole stillage. The stillage derived is direct to an anaerobic membrane bioreactor unit. The stillage is subjected to anaerobic digestion in the anaerobic digester and this produces mixed liquor that includes suspended solids. Mixed liquor including the suspended solids is directed to the membrane separation unit which filters the suspended solids and produces a concentrated reject (retentate) stream and a backset permeate stream. The backset permeate stream is then mixed with the incoming feedstock.

### FIELD OF THE INVENTION

The present invention relates to systems and methods for producing ethanol, and more particularly to systems and methods for producing both ethanol and biogas.

### BACKGROUND OF THE INVENTION

Ethanol producers face many challenges today, especially in the area of controlling energy costs and increasing production yield. Lately, with the constant increase in energy prices and a generally decreasing demand for the by-products of ethanol processes, conventional operational schemes are becoming less economical.

To reduce the water requirements of the fermentation process employed in an ethanol plant, thin stillage from a centrifuge or a solids separator is returned to the fermentation reactor or vessel. It is virtually impossible to remove 100% of the solids in a conventional centrifuge operation. This means that suspended solids, including what is referred to as non-active material, is returned to the fermentation reactor. When non-active material is returned to the fermentation reactors, this means that this limits the capacity of active material that can be added to each fermentation batch. Non-active material means material or matter that is nonfermentable or substantially nonfermentable.

t is known to utilize conventional completely stirred tank reactors for the anaerobic treatment of stillage. While these subsystems do produce energy from the backset stream, completely steered tank reactors inherently add biological mass to the material being treated. To insure the added solids do not inhibit the fermentation step, a pasteurization of the completely steered tank reactor effluent is required. This adds to cut process complexity and increases cost.

Therefore, there is a need for an ethanol system and process which will totally remove total suspended solids from whole or thin stillage which will in turn increase the effective capacity of each fermentation batch. In addition, there is a need in an ethanol system or process that will reduce the energy cost to produce the by-products such as dry distillers grain (DDG) and dry distillers grain syrup (DDGS). There is a need for systems and processes in an ethanol plant to generate a surplus of energy and potentially increase the overall production efficiency of an ethanol plant.

### SUMMARY OF THE INVENTION

The present invention relates to an ethanol production process that employees an anaerobic membrane bioreactor. Whole or thin stillage is directed into an anaerobic digester forming a part of the anaerobic membrane bioreactor. In the anaerobic digester, solids associated with the whole or thin stillage is anaerobically digested to form mixed liquor and biogas. The mixed liquor having a concentration of suspended solids is directed to a membrane separation unit that filters the mixed liquor and produces a concentrated reject stream (retentate) and a backset permeate that is virtually free of suspended solids. The backset permeate is directed back and mixed with incoming feedstock or substrate. Since the backset permeate is virtually free of suspended solids, it follows that the backset permeate does not contribute non-active suspended solids to the fermenter. This means that the suspended solids concentration in the backset fermenter does not limit or reduce the capacity of the fermenter.

In addition, the anaerobic digester produces biogas that can be utilized to power evaporators, dryers and other equipment that might be used in an ethanol process to produce by-products. Other objects and advantages of the present invention will become apparent and obvious from a study of the following description and the accompanying drawings which are merely illustrative of such invention.

### BRIEF DESCRIPTION OF THE DRAWINGS

FIG. 1 is a schematic illustration of the ethanol plant and method of producing ethanol and by-products.

FIG. 2 is a schematic view of an alternate ethanol production process.

FIG. 3 is a schematic of yet another alternate ethanol production process.

### DESCRIPTION OF EXEMPLARY EMBODIMENTS

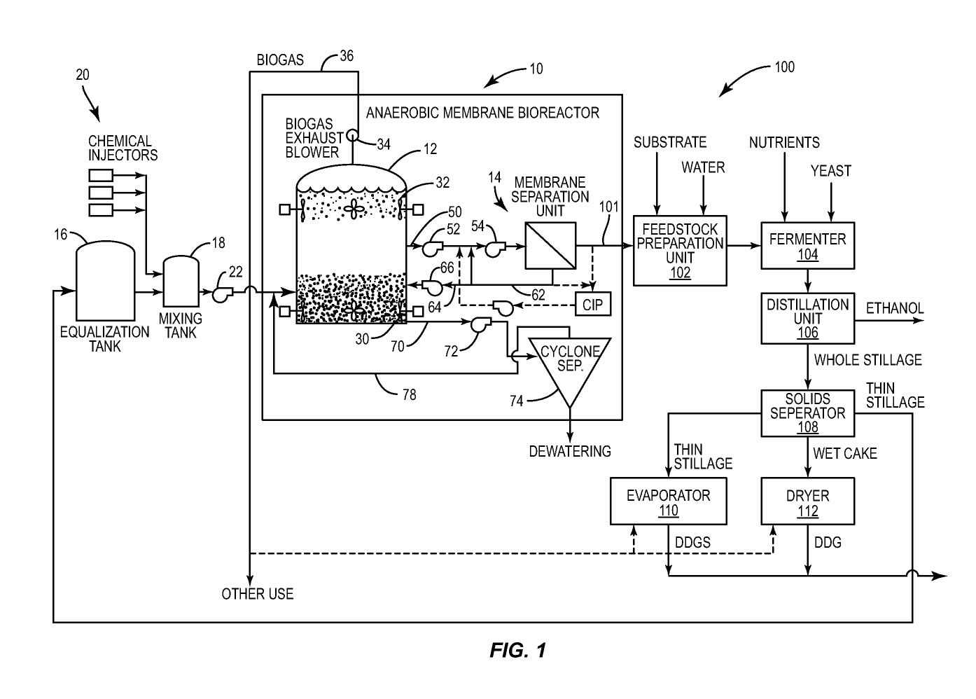

With further reference to the drawings, an ethanol plant or ethanol production system is shown therein and indicated generally by the numeral 100. Before discussing the process of producing ethanol, it might be beneficial to review basic system components for the plant. With reference to FIG. 1, there is provided a substrate or feedstock preparation unit 102. As will be discussed subsequently herein, feedstock such as corn is directed into the substrate preparation unit 102 and starches forming a part of the feedstock is converted to sugar. Downstream of the substrate preparation unit 102 is a fermenter or fermentation unit 104. Again, as will be discussed in more detail below, the fermentation unit 104 converts the sugar into ethanol and produces a beer that includes ethanol and other dissolved and suspended solids. Downstream of the fermentation unit 104 is a distillation unit 106 which functions to separate the ethanol from the beer and, as FIG. 1 indicates, produces ethanol and what is termed whole stillage. The whole stillage produced by the distillation unit 106, in one embodiment, is directed to a solids separator 108 such as a centrifuge unit. In the centrifuge unit 108, the whole stillage is separated into thin stillage and wet cake. The wet cake produced by the solids separator 108 is directed to a dryer 120 which functions to convert the wet cake to dry distillers grain (DDG). In the embodiment illustrated in FIG. 1, the thin stillage is split into two streams, one stream is directed to an evaporator 110 and the other thin stillage stream is directed to an anaerobic membrane bioreactor indicated generally by the numeral 10. The wet cake produced by the solids separator 108 is directed to a dryer 120 which functions to convert the wet cake to dry distillers grain (DDG). As shown in FIG. 1, the thin stillage stream directed to the anaerobic membrane bioreactor 10 passes through an equalization tank 16 and a mixing tank 18. A more comprehensive discussion of the treatment of stillage, whole or thin, by the anaerobic membrane bioreactor 10 will be forthcoming. Evaporator 110 on the other hand converts the thin stillage to dry distillers grain syrup (DDGS).

The processes carried out in the substrate preparation unit 102, fermenter 104 and distillation unit 106 are generally known and used in conventional ethanol production processes. A brief discussion of the basic processes that take place in these units may be beneficial. As just stated, there are various types of preparation steps or processes. Generally, however, a feedstock, along with water, is fed into the preparation unit 102. Various substrates or various type of plant materials can be used to produce ethanol. For example, common feedstocks include corn (dry meal or wet meal), wheat and sugar cane. Other types of plant material can also be used as the feedstock. Generally, the feedstock and water is heated and this can be achieved by adding steam to the feedstock—water mixture. The presence of the feedstock plus water with the addition of enzymes causes the starch found in the plant material to be converted to sugar. Thus, the substrate preparation unit 102 produces feedstock in the form of a mash where the starch associated with the feedstock has been converted to sugar.

The feedstock mash produced in the substrate preparation unit 102 is conveyed to the fermenter 104. Typically, yeast and nutrients are added to the feedstock mash. Generally the yeast converts the sugar to ethanol and in the process of fermentation, beer is produced. Beer includes ethanol and solids, particularly solids that are not reduced or broken down in the fermentation process. Generally, fermentation occurs at slightly higher temperatures than is typically found at ambient. In the fermentation process, as alluded to above, the yeast ferments the free sugar to ethanol and this fermentation process is carried out until substantially all of the sugars are consumed. The resulting beer includes alcohol and feedstock solids that are not fermentable, that is feedstock solids that cannot be digested by the yeast. In addition, the beer may include various substances that result from the breakdown of the feedstock or plant material such as acids, proteins, salts, oils and other dissolved solids.

The beer produced in the fermenter 104 is directed to the distillation unit 106. Here the beer is subjected to a distillation process where ethanol vapors are concentrated and separated from the feedstock solids. Various types of conventional distillation processes can be carried out to produce high purity ethanol. In order to obtain 100% pure ethanol that is required for some application, the ethanol can be further purified by a dehydration process. A typical dehydration process is performed using a molecular sieve as a desiccant. When the ethanol has been separated from the beer, the remaining composition is generally termed whole stillage.

The whole stillage in the embodiment shown in FIG. 1 is directed to the solids separator or centrifuge unit 108 which, in conventional fashion, separates the whole stillage into thin stillage and wet cake. In the case of the embodiment shown in FIG. 1, the thin stillage produced by the solids separator 108 is split into two streams, one stream is directed to the anaerobic membrane bioreactor 10 via the equalization tank 16 and mixing tank 18. As will be described below, the thin stillage is treated in the anaerobic digester or reactor 12 of the anaerobic membrane bioreactor 10. The anaerobic reactor 12 anaerobically digests the thin stillage to produce biogas and mixed liquor. The mixed liquor produced in the anaerobic reactor 12 is directed to the membrane separation unit 14 which filters the mixed liquor and produces a concentrated reject stream that is returned to the anaerobic reactor and a backset permeate stream that is substantially free of dissolved solids. The backset permeate stream, which is substantially free of non-active material (non-fermentable solids), is directed back to the preparation unit 102 or, in some embodiments, could be directed to the fermenter 104.

The thin stillage directed to the anaerobic membrane bioreactor 10 comprises a liquid that includes suspended solids that are generally not fermentable, as well as other soluble compositions such as oils, organic acids, salts, proteins and other materials that may inhibit yeast activity. Anaerobic reactor 12 contains microorganisms that operate anaerobically (in the absence of oxygen) to break down the nonfermentable solids and other organic substances. The breakdown of these components produces biogas and treated stillage that is referred to herein as mixed liquor.

Turning now to the anaerobic membrane bioreactor 10, as noted above, the bioreactor includes an anaerobic reactor 12 designed to provide mechanical mixing in the bottom portion of the reactor and mechanical mixing in an upper or top portion of the reactor. In one embodiment there is no mechanical mixing or relatively little mixing in the intermediate or middle portion of the anaerobic reactor 12. In the reactor 12, heavy solids including larger biological floc and inorganic precipitated solids that form tend to settle to the bottom portion of the reactor and are mixed with the mixed liquor therein by the mixing that takes place in the bottom or lower portion of the reactor. Other lighter or finer solids tend to float to the upper portion of the reactor where the mechanical mixing that takes place maintains these solids in suspension in the top portion of the reactor. This tends to stratify the mixed liquor in the anaerobic reactor 12 into three distinct zones. That is, the concentration of solids in the intermediate portion of the reactor is lower compared to the concentration of solids in the bottom or upper portion of the reactor.

Located upstream of the anaerobic membrane bioreactor 10 is an equalization tank 16. Equalization tank 16 includes one or more mixers. In the case of the embodiment shown in FIG. 1, thin stillage separated by the solids separator 108 is directed into the equalization tank 16 and, in some embodiments, the thin stillage can be mixed in the equalization tank. Disposed downstream of the equalization tank 16 is the mixing tank 18. Mixing tank 18 preferably includes one or more internal mixers. Associated with the mixing tank 18 is one or more chemical injectors indicated generally by the numeral 20. Chemical injectors 20 function to inject various chemicals into the mixing tank 18 which are then mixed with the stillage. Various chemicals can be injected into the mixing tank 18 depending upon the conditions and makeup of the thin stillage. For example, it may be desirable to control the pH throughout the process, and in that case a caustic such as NAOH can be injected and mixed into the waste stream. Other chemicals such as iron salts, necessary mineral elements for anaerobic production of biogas, for example, can be added if desired. In some embodiments, the mixing tank 18 may be unnecessary. In this case, if chemicals are desired, they can be injected into a line or conduit through which the stillage passes.

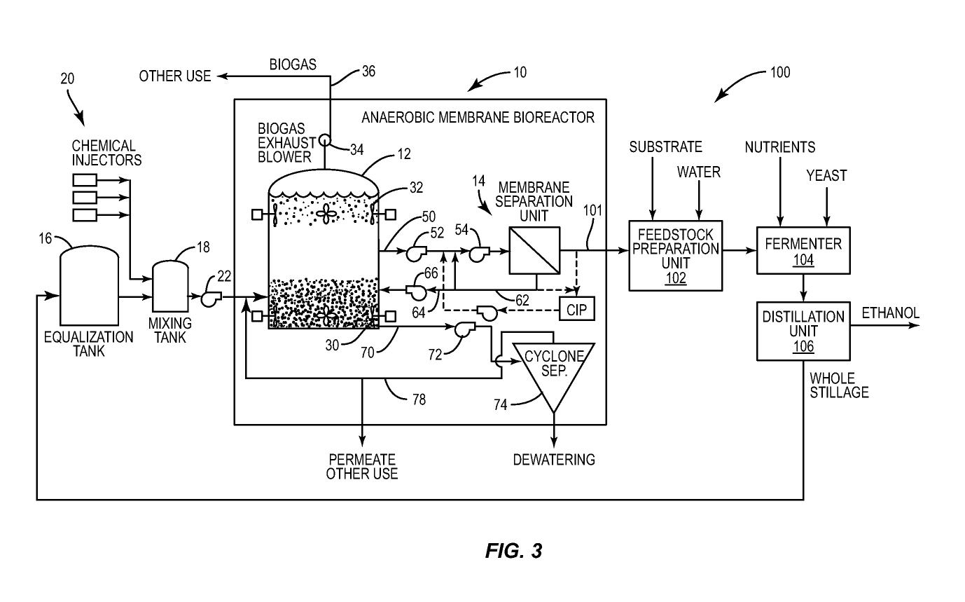

Stillage contained in the mixing tank 18 is directed into the anaerobic reactor 12. Anaerobic reactor 12 is a closed system designed to maintain anaerobic conditions within the reactor. Anaerobic reactor 12 can be of various sizes and capacities. The thin stillage, or whole stillage in the case of the embodiment shown in FIG. 3, is mixed with existing material or matter in the reactor to form mixed liquor. Generally the biodegradable components in the waste stream react with anaerobic biomass and reduce the amount of biodegradable solids associated with the stillage. In the process, biogas is produced as well as additional biological solids. The term “mixed liquor” is used herein includes, but is not limited to, a mixture of organic and inorganic solids, including biomass, biodegradable and non-biodegradable waste, water and biogas. Mixed liquor may reside in the reactor or be fed into the reactor as a recycle stream from the membrane separation unit 14. As previously alluded to, anaerobic reactor 12 is designed to stratify the mixed liquor.

Strategically placed in the anaerobic reactor is a series of mixers. First, there is one or more mixers 30 located in the bottom or lower portion of the reactor. Further, there is one or more mixers 32 located in the top or upper portion of the reactor 12. Thus, it is appreciated that in one embodiment there are no mixers located in the intermediate or middle region of the anaerobic reactor 12. Mixing the mixed liquor in the lower and upper portions of the reactor 12 improves and enhances reactions between the anaerobically digested components and the anaerobic biomass. Furthermore, for example, the mixing in the upper portion of the reactor prevents the solids from forming a blanket in the upper portion of the reactor 12.

Mixers 30 and 32 provide a mixing action, resulting in the bottom and top portion of the anaerobic reactor 12 being completely mixed. Various types of mixers can be used. In one embodiment the mixers are what is referred to as sidewall mounted mixers. These mixers project through the sidewall of the anaerobic reactor 12 with the propeller or mixing portion of the mixers being disposed internally within the reactor 12. Mixers 30 and 32 are generally uniformly spaced so as to provide uniform mixing of the mixed liquor in the top and bottom portions of the reactor. Although mechanical mixers are discussed and shown in the drawings, other types of conventional anaerobic reactor mixers can be used. For example, mixing can be accomplished by gas injection, mechanical streams, and mechanical pumps.

The depth and precise location of the stratified layers in the anaerobic reactor 12 can vary. In the way of an example, assume that the anaerobic reactor 12 is approximately 50 feet high. In such a case the bottom mixers 30 could be centered at approximately 3 feet from the bottom of the anaerobic reactor. Upper mixers 32 could be centered at approximately 38 feet from the bottom of the anaerobic reactor. In this case, at a height of 20 to 25 feet from the bottom of the anaerobic reactor, at least a portion of the intermediate or middle zone 40 would be located. Thus, in this example, line 50, which feeds mixed liquor from the anaerobic reactor 12 to the membrane separation unit 14, would be plumbed into the wall of the anaerobic reactor 12 at an intermediate point between 20 and 25 feet from the bottom of the anaerobic reactor. At this point the mixed liquor pumped from the anaerobic reactor would likely have a solids concentration less than the mixed liquor disposed in the bottom of the reactor.

Digesting solids associated with the stillage produces biogas. Biogas produced in the lower mixing zone will rise through the height of the reactor 12 and provide a gentle low shear mixing of the mixed liquor in the intermediate zone. Reactor 12 is provided with a biogas outlet that can pass by the force created by the biological production of biogas or can be enhanced through the utilization of an exhaust blower 34 and a biogas outlet 36. The biogas produced in the anaerobic reactor 12 can be utilized as a fuel source for various components employed in the ethanol plant. For example, biogas produced by the anaerobic reactor 12 can be utilized to provide a fuel source for the evaporator 110 and dryer 112. See FIG. 1.

As appreciated by those skilled in the art, the anaerobically biodegradable material contained in the stillage is digested through reactions in the reactor 12 where anaerobic (and facultative!) bacteria and methanogenic archaea convert the biodegradable stillage material to biogas which is substantially made up of methane and carbon dioxide and other lesser amounts of other elements in gaseous form such as hydrogen sulfide. These gaseous components are generally referred to herein as “biogas”. Biogas may also contain small amounts of water vapor, ammonia, and traces of other volatile compounds which may be present in the waste stream or formed during biodegradation. Resulting composition of the biogas by volume percent will vary depending on the particular digestible organics being processed. Preferred methane levels in biogas formed in the reactor 12 are in the range of about 50 to about 90 volume percent. Preferred carbon dioxide levels are in the range of about 5 to about 45 percent (by volume) and hydrogen sulfide levels can range from about 200 ppm (volume) to about 3 percent by volume.

Downstream from the anaerobic reactor 12 is the membrane separation unit 14. Mixed liquor from the anaerobic reactor 12 is directed to the membrane separation unit 14. Mixed liquor is taken from the intermediate or middle zone of the anaerobic reactor 12. This means that the mixed liquor directed from the anaerobic reactor 12 to the membrane separation unit includes a solids concentration less than would typically be found in the mixed liquor in the bottom or top portion of the anaerobic reactor 12. As seen in FIGS. 1-3, a membrane feed line is operatively interconnected between the anaerobic reactor 12 and the membrane separation unit 14 and serves to direct or channel mixed liquor from the reactor to the membrane separation unit. Various means can be employed for conveying mixed liquor from the reactor 12 to the membrane separation unit 14. In the exemplary embodiments shown in FIGS. 1-3, a membrane feed pump 52 is operably connected in feed line 50. Pump 52 pumps mixed liquor from the reactor through line 50 to the membrane separation unit 14. The membrane feed pump 52 provides a base line pressure, in this embodiment, to the membrane separation unit 14. In one embodiment, the membrane separation unit 14 is a continuously recirculated hydraulic loop that includes membrane modules, a membrane recirculation pump 54 and conventional membrane performance controls. The membrane recirculation pump 54 pumps the mixed liquor in a constant recirculation loop around the membrane separation unit 14 to provide necessary cross-flow velocity.

Membrane separation unit 14 filters or separates the mixed liquor into two streams, a reject or retentate stream that is relatively concentrated and a backset permeate stream that is substantially free of suspended solids. The concentrated reject or retentate stream is directed from the membrane separation unit 14 through a reject line 62. Reject line 62 is operative to recycle the reject or retentate stream back to the membrane feed pump 52 or back to the anaerobic reactor 12. That is, in one embodiment, at least a portion of the reject stream is returned to the anaerobic reactor 12 and mixed with the mixed liquor therein. This is achieved through return line 64. Thus, as noted above, a portion of the reject stream is taken off the recycled line 62 and returned via recirculation pump 66 to the anaerobic reactor 12. In one embodiment, the pump 66 is replaced with a flow control valve and the force required to return the reject stream to the reactor is provided by the membrane feed pump, pump 52.

The backset permeate stream produced by the membrane separation unit 14 is returned via line 101 to the substrate preparation unit 102 or in some cases directly to the fermenter 104. It should be appreciated that the backset permeate in relatively free of suspended solids and what is referred to as non-active fermentable material. This means that suspended solids that are non-fermentable are not returned to the fermenter 104 and does not occupy space and capacity in the fermenter. This effectively enables the capacity of the fermenter to be increased and that in turn increases the overall efficiency of producing ethanol.

Table 1 below shows typical concentrations for total solids (TS), total dissolved solids (TDS), and total suspended solids (TSS) for three different substrates, corn to ethanol/wet mill, cellulosic ethanol, and corn to ethanol/dry mill. Note the substantial reduction in total suspended solids in the backset permeate for each of the substrates. In the case of corn to ethanol/wet mill substrate, for example, the total suspended solids in the thin stillage was 14,190 mg/L. The membrane bioreactor including the anaerobic reactor 12 and the membrane separation unit 14 was effective to reduce the total suspended solids to only 470 mg/L in the backset permeate, a reduction of approximately 97%. There were similar substantial reductions in total suspended solids for the other two substrates shown in Table 1. As Table 1 also indicates, there were even substantial reductions in total dissolved solids.

TABLE I
Solids Summary for Different Ethanol Plants and Stillages

| | Plant | | |
|---|---|---|---|
| | Corn-to-Ethanol/Web Mill | | |
| Stream | Thin Stillage | Backset Permeate | % Reduction |
| TS (mg/L) | 64,000 | 10,300 | 84 |
| TDS (mg/L) | 49,810 | 9,830 | 80 |
| TSS (mg/L) | 14,190 | 470 | 97 |
| | Plant | | |
| | Cellulosic Ethanol | | |
| Stream | Whole Stillage | Backset Permeate | % Reduction |
| TS (mg/L) | 91,600 | 8,900 | 90 |
| TDS (mg/L) | 25,850 | 8,400 | 68 |
| TSS (mg/L) | 65,750 | 500 | 99 |
| | Plant | | |
| | Corn-to-Ethanol/Dry Mill | | |
| Stream | Thin Stillage | Backset Permeate | % Reduction |
| TS (mg/L) | 83,700 | 8,170 | 90 |
| TDS (mg/L) | 47,700 | 8,070 | 83 |
| TSS (mg/L) | 36,000 | 100 | 100 |

The anaerobic membrane bioreactor 10 also includes a clean in place (CIP) unit. The clean in place unit is a system or unit that is operative to periodically, or from time-to-time, clean the membrane separator unit 14 by backwashing the respective membranes that make up the membrane separation unit. Various membrane cleaning systems can be employed. In one example, the clean in place unit is designed to utilize the retentate from the membrane separator unit 14 to backwash and clean the respective membranes of the membrane separation unit. Details of the clean in place unit are not dealt with herein because such systems or units and how they operate are well known and appreciated by those skilled in the art.

The anaerobic membrane bioreactor in one embodiment may include a system and process for removing solids from the anaerobic reactor 12. The anaerobic membrane bioreactor 10 also includes a system and process for removing solids from the anaerobic reactor 12. More particularly, there is a solids separation process that includes a solids separator 74 such as a hydrocyclone separator. The solids separator 74 is designed to preferentially separate heavy solids which include a relatively high percentage of inorganic precipitants, from the lighter solids which typically include a relatively high concentration of biomass. As noted above, solids are removed from the anaerobic reactor 12 in order to maintain or control sludge retention time (SRT). In addition, there can be a substantial buildup of heavy inorganic solids within the anaerobic reactor 12 and these solids can be removed by directing them from the anaerobic reactor to a solids separator. In any event, there are various ways of removing solids from the anaerobic membrane bioreactor 10. For example, in one embodiment, solids can simply be wasted from the anaerobic reactor 12 in conventional fashion. In another example, solids can be removed from the retentate stream leaving the membrane separation unit. In this case a selected or controlled amount of the retentate stream can be directed to a solids separator.

In the embodiment illustrated herein, solids are pumped from the lower portion of the anaerobic reactor 12 to a solids separator, which in the case of the example illustrated, is a hydrocyclone 74. In this regard, line 70 is operatively connected to the anaerobic reactor 12 and includes a pump 72. Line 70 and pump 72 are operatively connected to the solids separator 74 for directing mixed liquor including solids to the solids separator. Note that line 70 is connected to the reactor 12 such that mixed liquor is pulled from the bottom portion of the reactor 12. This, as explained above, is where the heavier solids are contained. In any event, the mixed liquor is pumped from the bottom portion of the reactor 12 through line 70 into the solids separator 74. Solids separator 74 produces an underflow which comprises solids that are heavier in nature and an overflow which comprises solids which are lighter in nature than the underflow. The overflow is pumped or fed through an overflow line 78 back to the anaerobic reactor 12 where it is mixed with the mixed liquor therein. The underflow or heavier solids produced by the solids separator or hydrocyclone 74 is directed through underflow line 76 for further treatment. For example, the heavier solids produced in the underflow can be directed to a dewatering unit for dewatering and further concentration.

The solids removal process just described with respect to the solids separator 74 can be operated in parallel with the membrane separation unit 14. In some instances, the solids removal process may be operated continuously while the membrane separation unit 14 is filtering mixed liquor from the reactor 12. In other cases the solids removal process may be operated intermittently in order to maintain a selected SRT. The SRT can vary depending on circumstances, and conditions. It is contemplated that the SRT for the embodiments illustrated and discussed herein can range from approximately 15 to approximately 80 days.

The solids separator 74 is not an essential component of the present invention. There are situations when the solids separator 74 is not required. More particularly, the solids separator 74 and the process of removing solids from the bottom portion of the anaerobic reactor 12 is useful when the influent stream or the feedwater stream includes a substantial amount of dissolved solids that precipitate when undergoing treatment in the process of the present invention. Some feedwater streams will not include substantial dissolved solids that will precipitate and in those cases the solids separation process utilizing the solids separator 74 may not be a requirement in the process of the present invention.

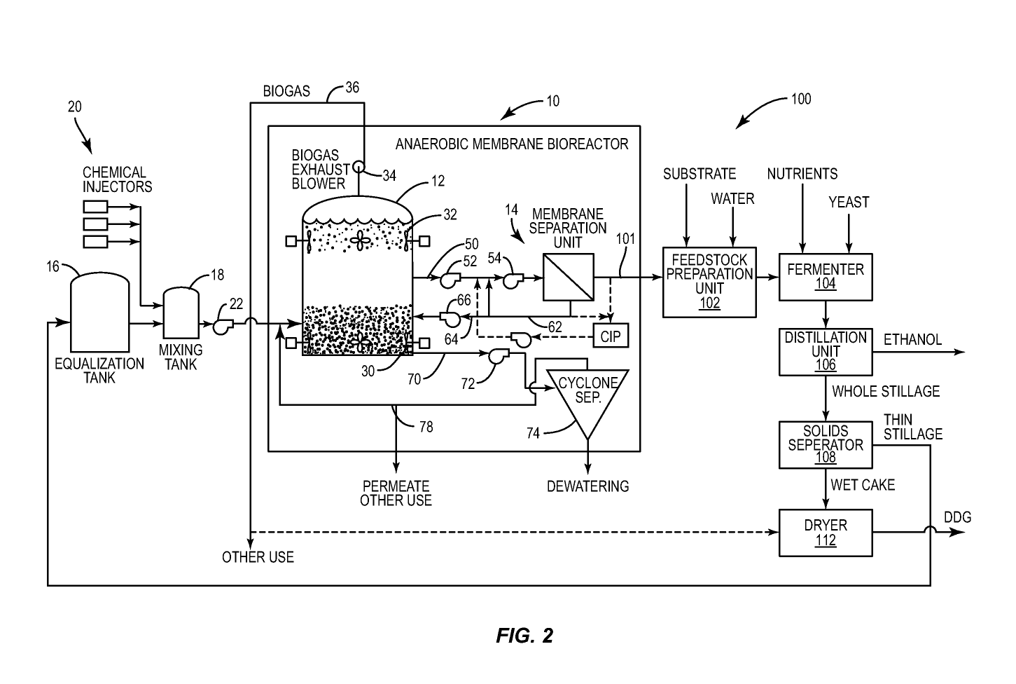

In the FIG. 1 embodiment, discussed above, the ethanol plant or system includes the evaporator 110 for treating thin stillage and a dryer 120 for treating the wet cake. The embodiment shown in FIG. 2 is substantially similar to the system and process shown in FIG. 1 and described above. FIG. 2 basically differs from FIG. 1 inasmuch as the FIG. 2 process does not include the evaporator 110 which receives a stream of thin stillage. However, as seen in FIG. 2, the anaerobic membrane reactor 10 does receive and treat a stream of thin stillage separated by the solids separator 108.

The FIG. 3 embodiment differs from the FIG. 1 embodiment inasmuch as the whole stillage produced by the distillation unit 106 is directed to the anaerobic membrane bioreactor 10. In the FIG. 3 design, there is no evaporator or dryer for treating the thin stillage and wet cake. However, the basic concepts described with respect to the embodiment of FIG. 1 apply to the embodiments shown in FIGS. 2 and 3. That is, the anaerobic membrane bioreactor is operative to digest non-fermentable solids to produce the mixed liquor and the membrane separation unit is operative to filter the mixed liquor to remove substantially all suspended solids such that the backset permeate stream produced by the membrane separation unit 14 is substantially free of suspended solids and which can be directed to the feedstock preparation unit 102 or in some cases directly to the fermenter 104.

The present invention may, of course, be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

1. A method of producing ethanol and biogas comprising:
(a) directing an incoming feedstock to a pretreatment area and pretreating the feedstock to free sugar from the feedstock;
(b) fermenting the sugar produced from the feedstock in a fermenter to produce beer which includes the feedstock and ethanol;
(c) distilling the beer to produce ethanol and whole stillage which includes liquid, dissolved solids and suspended solids;
(d) directing at least a portion of the whole stillage or a portion of thin stillage produced from the whole stillage to an anaerobic membrane bioreactor having an anaerobic digester and a membrane separation unit and:
(1) in the anaerobic digester anaerobically digesting solids in the stillage and producing biogas and a mixed liquor;
(2) directing the mixed liquor from the anaerobic digester to the membrane separation unit and removing suspended solids from the mixed liquor and in the process producing a concentrated reject stream and a backset permeate that is substantially free of suspended solids;
(e) recycling at least a portion of the backset permeate and mixing the backset permeate with the incoming feedstock and wherein the mixture of the backset permeate and the incoming feedstock is fermented in the fermenter;
(f) stratifying the mixed liquor in the anaerobic digester by forming a first lower mixed liquor zone where the mixed liquor in the first lower mixed liquor zone includes a relatively high concentration of solids, and forming a second mixed liquor zone above the first lower mixed liquor zone where the mixed liquor in the second zone includes a solids concentration substantially less than the concentration of solids in the first lower mixed liquor zone; and
(q) directing mixed liquor from the second mixed liquor zone in the anaerobic digester to the membrane separation unit where the mixed liquor is separated into the concentrated reject stream and the backset permeate that is relatively free of suspended solids.

2. (canceled)

3. The method of claim 1 including separating the whole stillage into the thin stillage and wet cake, and directing the thin stillage to the anaerobic digester and anaerobically digesting solids in the thin stillage and producing the biogas and the mixed liquor.

4. The method of claim 1 including directing the whole stillage to a centrifuge and separating thin stillage from the whole stillage, and directing at least a portion of the thin stillage to the anaerobic digester and anaerobically digesting solids in the thin stillage to produce the biogas and mixed liquor.

5. The method of claim 1 further including directing the whole stillage to a stillage separator and separating thin stillage from the whole stillage, and splitting the thin stillage into at least two streams, and directing one stream of the thin stillage to the anaerobic membrane bioreactor and anaerobically digesting solids in the thin stillage and producing the biogas and the mixed liquor, and directing the other thin stillage stream to an evaporator and producing dry distiller's grain (DDG).

6. (canceled)

7. The method of claim 1 including recycling at least a portion of the concentrated reject stream to the anaerobic digester and mixing the concentrated reject stream with the mixed liquor in the anaerobic digester; and directing the mixed liquor and solids from the first lower mixed liquor zone to a solids separator and separating the mixed liquor and solids into a heavier solids stream and a lighter solids stream containing biomass.

8. The method of claim 7 including recycling at least a portion of the lighter solids stream containing biomass to the anaerobic digester and mixing the lighter solids stream with the mixed liquor in the anaerobic digester.

9. The method of claim 1 including providing a mixing action in the first lower mixed liquor zone and mixing the mixed liquor and solids therein; and maintaining the mixed liquor in the second zone in an unmixed state or in a state where the mixing action in the second zone is substantially less than the mixing action in the first lower mixed liquor zone.

10. The method of claim 1 wherein the method includes forming a third mixed liquor zone over the second mixed liquor zone; and wherein both the first and third mixed liquor zones are mixed with relatively heavy solids residing in the first mixed liquor zone and relatively light solids residing in the third mixed liquor zone.

11. The method of claim 7 wherein the solids separator includes a hydrocyclone.

12. The method of claim 7 wherein during certain time intervals both the membrane separation unit and the solids separator are operated simultaneously and wherein there is one mixed liquor flow from the anaerobic digester to the membrane separation unit and another mixed liquor flow from the anaerobic digester to the solids separator, and wherein the two flows are independent of each other.

13. The method of claim 1 wherein the first lower mixed liquor zone is mixed with mechanical mixers; and wherein the second mixed liquor zone above the first lower mixed liquor zone is unmixed.

14-19. (canceled)

20. The ethanol plant of claim 15 wherein the membrane separation unit includes one or more nanofiltration or reverse osmosis membranes.

21. (canceled)

22. A method of producing ethanol and biogas comprising:
(a) directing an incoming feedstock to a pretreatment area and pretreating the feedstock to free sugar from the feedstock;
(b) fermenting the sugar produced from the feedstock in a fermenter to produce beer which includes the feedstock and ethanol;
(c) distilling the beer to produce ethanol and whole stillage which includes liquid, dissolved solids and suspended solids;
(d) directing the whole stillage to an anaerobic membrane bioreactor having an anaerobic digester and a membrane separation unit and:
(1) in the anaerobic digester anaerobically digesting solids in the whole stillage and producing biogas and a mixed liquor;
(2) directing the mixed liquor from the anaerobic digester to the membrane separation unit and removing suspended solids from the mixed liquor and in the process producing a concentrated reject stream and a backset permeate that is substantially free of suspended solids; and
(e) recycling at least a portion of the backset permeate and mixing the backset permeate with the incoming feedstock and wherein the mixture of the backset permeate and the incoming feedstock is fermented in the fermenter.

23. The method of claim 22 including:
stratifying the mixed liquor in the anaerobic digester by forming a first lower mixed liquor zone where the mixed liquor in the first lower mixed liquor zone includes a relatively high concentration of solids, and forming a second mixed liquor zone above the first lower mixed liquor zone where the mixed liquor in the second zone includes a solids concentration substantially less than the concentration of solids in the first lower mixed liquor zone; and
directing mixed liquor from the second mixed liquor zone in the anaerobic digester to the membrane separation unit where the mixed liquor is separated into the concentrated reject stream and the backset permeate that is relatively free of suspended solids.

24. The method of claim 23 including recycling at least a portion of the concentrated reject stream to the anaerobic digester and mixing the concentrated reject stream with the mixed liquor in the anaerobic digester; and directing the mixed liquor and solids from the first lower mixed liquor zone to a solids separator and separating the mixed liquor and solids into a heavier solids stream and a lighter solids stream containing biomass.

25. The method of claim 24 including recycling at least a portion of the lighter solids stream containing biomass to the anaerobic digester and mixing the lighter solids stream with the mixed liquor in the anaerobic digester.

26. The method of claim 22 wherein the whole stillage produced by distilling the beer is not subject to treatment in a solids separator prior to being directed to the anaerobic membrane bioreactor.
